(19) United States
(12) Patent Application Publication
(10) Pub. No.: US 2015/0112322 A1
(43) Pub. Date: Apr. 23, 2015

(54) ELECTROSURGICAL INSTRUMENT AND SYSTEM

(71) Applicant: GYRUS MEDICAL LIMITED, Cardiff (GB)

(72) Inventor: David Williams, Caerphilly (GB)

(21) Appl. No.: 14/404,579

(22) PCT Filed: Jun. 6, 2013
(86) PCT No.: PCT/GB2013/051493

§ 371 Date: Nov. 28, 2014

(30) Foreign Application Priority Data
Jun. 12, 2012 (GB) 1210297.6

(51) Int. Cl. A61B 18/12

(52) U.S. Cl. CPC A61B 18/1206; A61B 2018/1226; A61B 2018/1286; A61B 2018/00178
USPC 606/34

(57) ABSTRACT

A handheld electrosurgical instrument includes a handpiece (2) and an electrode assembly (3), the electrode assembly including one or more electrodes (12), (13) and a connector (8), (9) by which the electrode assembly (3) is capable of being attached and detached with respect to the handpiece (2). The handpiece includes a battery (4), an RF oscillator circuit (30) for generating a radio frequency output, an RF output circuit (5) and switching means (7) operable by a user of the electrosurgical instrument. A control circuit is provided (6) for controlling the RF output, the control circuit (6) being capable of receiving signals from the switching means (7) and causing one or more RF outputs to be supplied to the electrode assembly (3) in response thereto. The RF output circuit includes a transformer including at least one primary winding (14) and a plurality of secondary windings (15), (16), (17). The connector on the electrode assembly (9) includes a component (29) capable of physically selecting a particular secondary winding or combination of secondary windings so as to modify the RF output reaching the one or more electrodes.

### TECHNICAL FIELD

This invention relates to an electrosurgical instrument and to an electrosurgical system for use in the treatment of tissue. Such systems are used in endoscopic or “keyhole” surgery, as well as more traditional “open” surgery.

### BACKGROUND TO THE INVENTION

### Prior Art

Many electrosurgical systems have some form of identification system, such that when an electrosurgical instrument is connected to an electrosurgical generator, the generator is able to detect which type of instrument is present, and even use settings such as power and voltage settings which are appropriate for that particular instrument or type of instrument. Our U.S. Pat. No. 6,074,386 is one example of such an identification system, although other types are also known.

### Summary of the Invention

The present invention attempts to provide an alternative to such identification systems, such that the generator used as part of the electrosurgical system can be substantially simplified, which is particularly important when attempting to provide a handheld electrosurgical instrument. Accordingly, a handheld electrosurgical instrument comprises a handpiece and an electrode assembly, the electrode assembly including one or more electrodes and a connector by which the electrode assembly is capable of being attached and detached with respect to the handpiece, the handpiece including,

a) a battery;

b) an RF oscillator circuit for generating a radio frequency output;

c) an RF output circuit including a transformer including at least one primary winding and a plurality of secondary windings,

c) a switching means operable by a user of the electrosurgical instrument; and

d) a control circuit for controlling the RF output, the control circuit being capable of receiving signals from the switching means and causing one or more RF outputs to be supplied to the RF output circuit in response thereto;

wherein the connector on the electrode assembly includes a component capable of physically selecting a particular secondary winding or combination of secondary windings so as to modify the RF output reaching the one or more electrodes.

Conveniently, the connector includes one or more pins, a characteristic of the one or more pins causing the selection of a particular secondary winding or combination of secondary windings. This arrangement is different from identification systems such as that disclosed in pending US patent application 2010/0305563 which uses pins on a connector as a way for the generator identify the instrument connected thereto. In the present arrangement, the pins physically select the secondary winding or combination of windings rather than identifying the instrument.

The characteristic of the one or more pins causing the selection of a particular secondary winding or combination of secondary windings conveniently comprises the position of the one or more pins. Alternatively, the characteristic of the one or more pins causing the selection of a particular secondary winding or combination of secondary windings comprises the length of the one or more pins. In either arrangement, the one or more pins can act as a “jumper” to make or break an electrical connection within the handpiece depending on their position or length. This connection (or absence of a connection) causes the selection of the particular secondary winding or combination of secondary windings. In this way, the RF waveform produced from the RF output circuit is selected so as to be suitable for the particular electrode assembly connected to the handpiece.

In one convenient arrangement, the connector on the electrode assembly includes a plurality of contacts, and the one or more pins are jumper pins adapted to form an electrical short between a selected combination of the plurality of contacts. Alternatively, the one or more pins cause the operation of a relay which selects a particular secondary winding or combination of secondary windings. As before, the relay can be activated by the one or more pins acting to make or break an electrical connection within the handpiece depending on their position or length.

From another aspect, embodiments of the invention also provide an electrosurgical instrument comprising a handpiece and an electrode assembly releasably connectable to the handpiece, the handpiece comprising: an RF signal source; a control switch operable by the user to control the handpiece to output a radio frequency (RF) signal derived from the RF signal source; and an output interface comprising output selector circuitry and output signal generation circuitry, the output selector circuitry receiving respective different RF output signals having different properties obtained from the output signal generation circuitry, the output signal generation circuitry adapting the RF signal derived from the RF signal source into the different RF output signals; the electrode assembly comprising: one or more electrodes forming an end effector for the treatment of tissue when an RF treatment signal is applied thereto; and input interface circuitry arranged to interconnect with the output selector circuitry in the output interface so as to select one of the available different RF output signals from the output interface as the RF treatment signal.

With the above arrangement a handpiece that is able to conveniently provide multiple different outputs for use with different types of end effector can be obtained. Moreover, the interaction of the handpiece output interface and the electrode assembly input interface allows for the automatic selection of the appropriate RF output for the end effector.

In one embodiment the output signal generation circuitry in the output interface conveniently comprises a transformer including a primary coil and a plurality of secondary coils, the plurality of secondary coils providing the respective different RF output signals. Use of a transformer with multiple secondary windings is a convenient way to produce multiple output signals having different signal properties. In this respect, preferably the secondary coils have different numbers of windings.

In one embodiment the output selector circuitry has a plurality of control input terminals, and the input interface circuitry includes an electrical contact positioned so as to make contact with a selected one of the control input terminals in the output selector circuitry so as to cause the output selector circuitry to output a desired one of the available different RF output signals for use as the RF treatment signal. In particular, the output selector circuitry may be an electrical relay.

In an alternative embodiment the output selector circuitry comprises a plurality of output terminals respectively corresponding to the available different RF output signals, and the input interface circuitry comprises an electrical contact positioned so as to contact a desired one of the plurality of output terminals to obtain the RF output signal corresponding thereto for use as the RF treatment signal. Both arrangements provide for convenient output selection based on the physical position of the electrical contact.

According to an alternative aspect of the invention, an electrosurgical system is provided comprising a handpiece and a plurality of electrode assemblies, the electrode assemblies being at least of a first type or a second type of electrode assembly, and each including one or more electrodes and a connector by which the electrode assembly is capable of being attached and detached with respect to the handpiece, the handpiece comprising

a) a battery;

b) an RF oscillator circuit for generating a radio frequency output;

c) an RF output circuit including a transformer including at least one primary winding and a plurality of secondary windings,

c) a switching means operable by a user of the electrosurgical instrument; and

d) a control circuit for controlling the RF output, the control circuit being capable of receiving signals from the switching means and causing one or more RF outputs to be supplied to the RF output circuit in response thereto;

wherein the connector on the first type of electrode assembly includes a first component and the connector on the second type of electrode assembly includes a second component, the first and second components being capable of physically selecting a particular secondary winding or combination of secondary windings such that the particular secondary winding or combination of secondary windings selected for the first type of electrode assembly is different from the particular secondary winding or combination of secondary windings selected for the second type of electrode assembly.

In this way, the RF output circuit can be changed to provide an RF waveform suitable for either the first type of electrode assembly or the second type of electrode assembly, depending on the characteristic component present within the connector of each type of electrode assembly. This can be achieved by the component physically selecting the particular secondary winding or combination of secondary windings necessary to produce the desired output, rather than by the handpiece having to identify the particular type of electrode assembly attached to the handpiece and take corresponding action to adjust the output in response thereto.

For a first type of electrode assembly (say, for instance, a cutting needle), the one or more pins are such as to select a secondary winding such as to produce a suitable cutting voltage. For a second type of electrode assembly (say, for instance, a pair of coagulating jaws), the position or length of the one or more pins is different, so as to select a different secondary winding and produce a different output more suited to the coagulation of tissue.

### BRIEF DESCRIPTION OF THE DRAWINGS

The invention will now be described in more detail, by way of example only, with reference to the accompanying drawings, in which;

FIGS. 1 & 2 are schematic diagrams of handheld electrosurgical instruments in accordance with the present invention,

FIGS. 3 & 4 are a schematic diagrams of the RF output circuit and connectors for the handheld electrosurgical instrument of FIG. 1, and

FIGS. 5 to 7 are schematic diagrams of an alternative embodiment of RF output circuit and connectors for the handheld electrosurgical instrument of FIG. 2.

### DESCRIPTION OF THE EMBODIMENTS

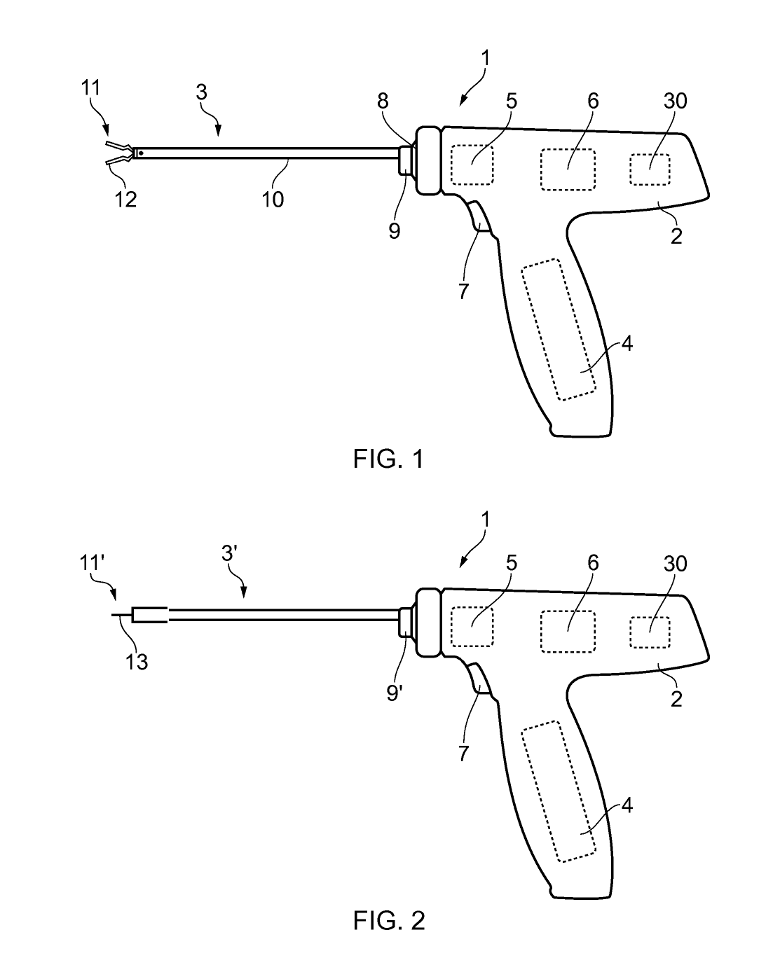

FIG. 1 shows a handheld electrosurgical instrument shown generally at 1, and comprising a handpiece 2 and an electrode assembly 3. The handpiece 2 includes a battery 4, RF oscillator circuit 30, RF output circuit 5 and control circuit 6. The handpiece is provided with a handswitch 7 for giving instructions to the control circuit 6. The handpiece includes a handpiece connector 8 mating with a corresponding electrode assembly connector 9 carried by the electrode assembly 3. The electrode assembly includes an elongate shaft 10 and an end effector 11 at the distal end of the shaft. In FIG. 1, the electrode assembly is such that the end effector 11 comprises a pair of jaws 12. Leads (not shown) run along the shaft 10 of the electrode assembly 3 to connect the RF circuit to the end effector 11, such that the jaws 12 constitute electrodes capable of the coagulation of tissue.

FIG. 2 shows the handpiece 2 together with a different electrode assembly 3′. The electrode assembly 3′ has a connector 9′ and is such that its end effector 11′ comprises a deployable cutting needle 13. Once again, leads (not shown) run along the shaft 10′ of the electrode assembly 3′ to connect the RF circuit to end effector 11′, such that the cutting needle 13 constitutes an electrode capable of the cutting of tissue.

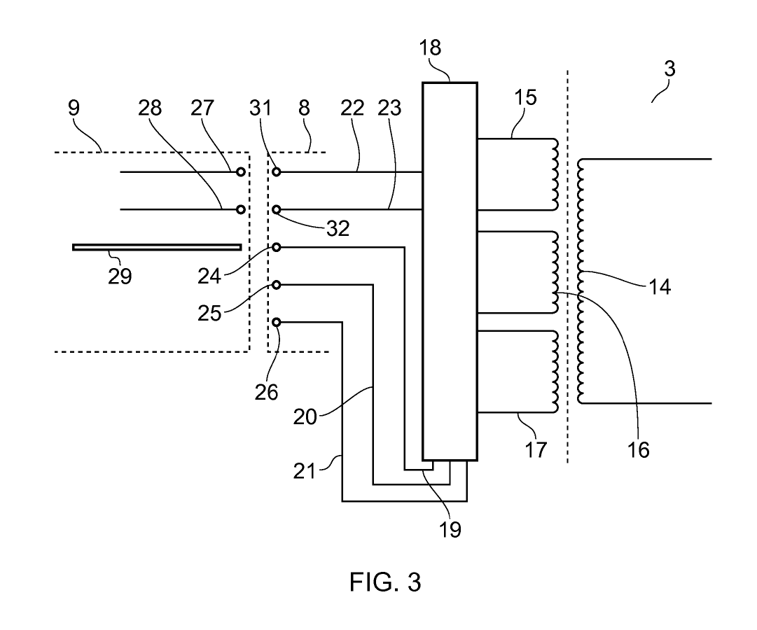

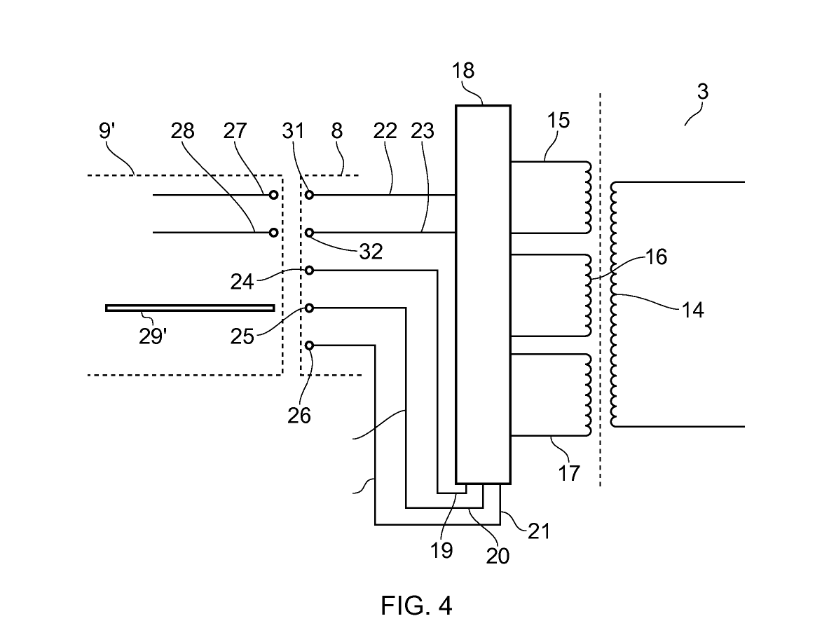

FIG. 3 shows the RF output circuit 3 and the connectors 8 and 9. The RF output circuit 3 includes a primary winding 14, and three secondary windings 15, 16 & 17. The number of turns on each of the secondary windings is different, such that whichever secondary winding is selected will produce a different out signal. The secondary windings are connected to a relay 18 driven by a power source (not shown), the relay being capable of selecting a particular secondary winding in response to signals from input lines 19, 20 & 21.

The handpiece connector 8 includes RF output lines 22 & 23, the RF output lines ending in ending in contacts 31 & 32. The handpiece connector also includes contacts 24, 25 & 26 connected respectively to the input lines 19, 20 & 21. The electrode assembly connector 9 includes RF connections 27 & 28, which mate with the RF output lines 22 & 23 when the connectors 8 & 9 are connected together. The electrode assembly connector also includes a contact pin 29, positioned to contact one of the contacts 24, 25 or 26 when the connectors 8 & 9 are connected together.

Consider the coagulating electrode assembly of FIG. 1. When the connectors 8 & 9 are mated together, the contact pin 29 makes contact with the contact 24 completing a circuit via line 19 to send a signal to the relay 18 which in turn selects secondary winding 15 to form part of the transformer with primary winding 14. This results in an RF waveform suitable for tissue coagulation being supplied to the RF output lines 22 & 23.

FIG. 4 shows the RF output circuit 3 and the connectors 8 and 9′ when the tissue-cutting electrode assembly of FIG. 2 is employed. All of the components are identical to those described with reference to FIG. 3, with the exception that a contact pin 29′ is located in a different position. When the connectors 8 & 9′ are mated one with another, the position of the contact pin 29′ means that it makes contact with contact 25 rather than contact 24. The circuit completed on line 25 sends a signal to the relay 18 to select secondary winding 16 to form part of the transformer with primary winding 14. This results in an RF waveform suitable for tissue cutting being supplied to the RF output lines 22 & 23.

In this way, the voltage of the RF output supplied to the end effectors 11 & 11′ is different, by virtue of the different secondary windings chosen by the relay 18 in each case. The RF output can therefor be matched to the particular electrode assembly used in conjunction with the handpiece 2, while maintaining the simplicity of the handpiece itself.

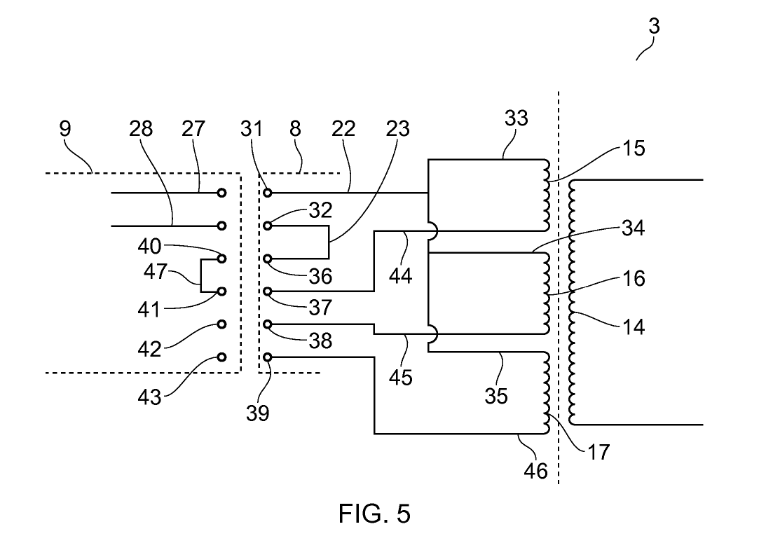

FIG. 5 shows an alternative embodiment in which the relay 18 is omitted. The primary winding 14 and secondary windings 15, 16 & 17 are as previously described, as are the output lines 22 & 23, and the RF connections 27 & 28. One side of each of the secondary windings 15, 16 & 17 is connected to the output line 22 via lines 33, 34 & 35 respectively. The handpiece connector 8 also has additional contacts 36, 37, 38 & 39 as will be described subsequently, and the electrode assembly connector 9 has corresponding contacts 40, 41, 42 & 43.

Output line 23 is connected to contact 32 as before, but in this embodiment also to contact 36 on the connector 8.

The other side of the secondary winding 15 is connected to contact 37 via line 44, while the other side of the secondary winding 16 is connected to contact 38 via line 45, and the other side of the secondary winding 17 is connected to contact 39 via line 46. In the connector 9, a jumper pin 47 shorts contacts 40 & 41 such that when the connector 9 mates with the connector 8, the secondary winding 15 is placed in communication across RF output lines 22 & 23.

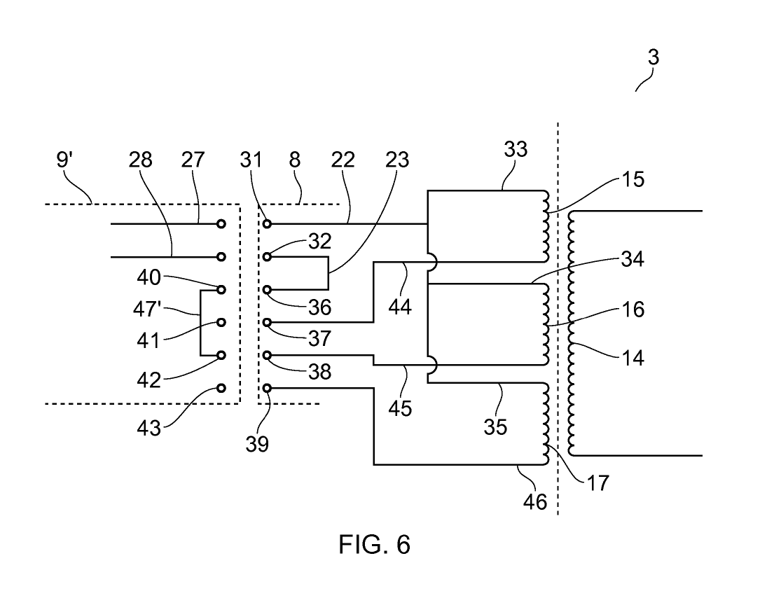

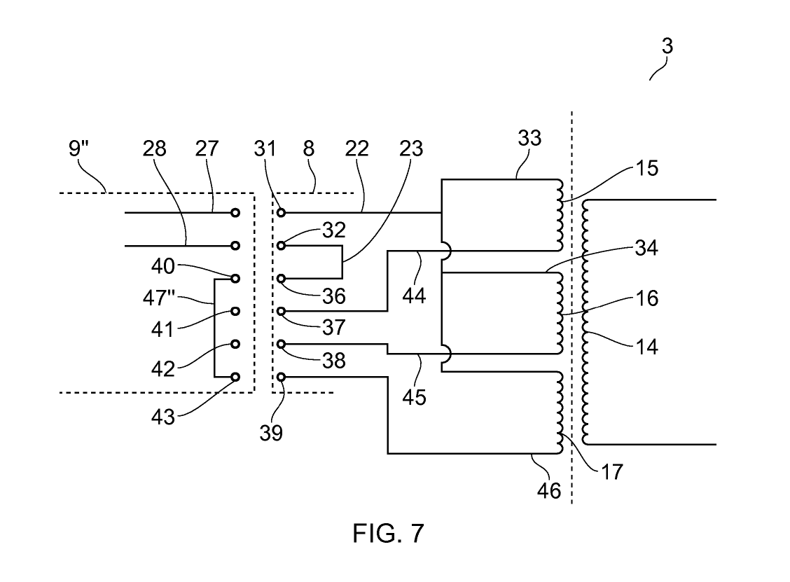

FIG. 6 shows an alternative connector 9′ in which the jumper pin 47′ shorts contacts 40 & 42. In this way when the connector 9′ mates with the connector 8, the secondary winding 16 is placed in communication across RF output lines 22 & 23. Similarly, FIG. 7 shows an alternative connector 9″ in which the jumper pin 47″ shorts contacts 40 & 43. In this way when the connector 9″ mates with the connector 8, the secondary winding 17 is placed in communication across RF output lines 22 & 23. Depending on the type of jumper pin present in the connector, a different secondary winding is selected for use as part of the output stage of the handpiece, such that the RF output produced is suitable for the particular type of electrode assembly being employed at that particular time. The RF output can therefore be matched to the particular electrode assembly used in conjunction with the handpiece 2, while maintaining the simplicity of the handpiece itself.

Various modifications may be made to the above described embodiments, whether by way of addition, deletion, or substitution of various components thereof, to provide additional embodiments any and all of which are intended to be encompassed by the appended claims.

1. A handheld electrosurgical instrument comprising a handpiece and an electrode assembly, the electrode assembly including one or more electrodes and a connector by which the electrode assembly is capable of being attached and detached with respect to the handpiece, the handpiece including,
a) a battery;
b) an RF oscillator circuit for generating a radio frequency output;
c) an RF output circuit including a transformer including at least one primary winding and a plurality of secondary windings,
c) a switching means operable by a user of the electrosurgical instrument; and
d) a control circuit for controlling the RF output, the control circuit being capable of receiving signals from the switching means and causing one or more RF outputs to be supplied to the RF output circuit in response thereto;
wherein the connector on the electrode assembly includes a component capable of physically selecting a particular secondary winding or combination of secondary windings so as to modify the RF output reaching the one or more electrodes.

2. An electrosurgical instrument according to claim 1, wherein the connector includes one or more pins, a characteristic of the one or more pins causing the selection of a particular secondary winding or combination of secondary windings.

3. An electrosurgical instrument according to claim 2, wherein the characteristic of the one or more pins causing the selection of a particular secondary winding or combination of secondary windings comprises the position of the one or more pins.

4. An electrosurgical instrument according to claim 2, wherein the characteristic of the one or more pins causing the selection of a particular secondary winding or combination of secondary windings comprises the length of the one or more pins.

5. An electrosurgical instrument according to claim 3, wherein the connector on the electrode assembly includes a plurality of contacts, and the one or more pins are jumper pins adapted to form an electrical short between a selected combination of the plurality of contacts.

6. An electrosurgical instrument according to claim 3, wherein the one or more pins cause the operation of a relay which selects a particular secondary winding or combination of secondary windings.

7. An electrosurgical system comprising a handpiece and a plurality of electrode assemblies, the electrode assemblies being at least of a first type or a second type of electrode assembly, and each including one or more electrodes and a connector by which the electrode assembly is capable of being attached and detached with respect to the handpiece, the handpiece comprising
a) a battery;
b) an RF oscillator circuit for generating a radio frequency output;
c) an RF output circuit including a transformer including at least one primary winding and a plurality of secondary windings,
c) a switching means operable by a user of the electrosurgical instrument; and
d) a control circuit for controlling the RF output, the control circuit being capable of receiving signals from the switching means and causing one or more RF outputs to be supplied to the RF output circuit in response thereto;
wherein the connector on the first type of electrode assembly includes a first component and the connector on the second type of electrode assembly includes a second component, the first and second components being capable of physically selecting a particular secondary winding or combination of secondary windings such that the particular secondary winding or combination of secondary windings selected for the first type of electrode assembly is different from the particular secondary winding or combination of secondary windings selected for the second type of electrode assembly.

8. An electrosurgical instrument comprising a handpiece and an electrode assembly releasably connectable to the handpiece,
the handpiece comprising:
an RF signal source;
a control switch operable by the user to control the handpiece to output a radio frequency (RF) signal derived from the RF signal source; and
an output interface comprising output selector circuitry and output signal generation circuitry, the output selector circuitry receiving respective different RF output signals having different properties obtained from the output signal generation circuitry, the output signal generation circuitry adapting the RF signal derived from the RF signal source into the different RF output signals;
the electrode assembly comprising:
one or more electrodes forming an end effector for the treatment of tissue when an RF treatment signal is applied thereto; and
input interface circuitry arranged to interconnect with the output selector circuitry in the output interface so as to select one of the available different RF output signals from the output interface as the RF treatment signal.

9. An electrosurgical instrument according to claim 8, wherein the output signal generation circuitry in the output interface comprises a transformer including a primary coil and a plurality of secondary coils, the plurality of secondary coils providing the respective different RF output signals.

10. An electrosurgical instrument according to claim 9, wherein the secondary coils have different numbers of windings.

11. An electrosurgical instrument according to claim 8, wherein the output selector circuitry has a plurality of control input terminals, and the input interface circuitry includes an electrical contact positioned so as to make contact with a selected one of the control input terminals in the output selector circuitry so as to cause the output selector circuitry to output a desired one of the available different RF output signals for use as the RF treatment signal.

12. An electrosurgical instrument according to claim 11, wherein the output selector circuitry is an electrical relay.

13. An electrosurgical instrument according to claim 8, wherein the output selector circuitry comprises a plurality of output terminals respectively corresponding to the available different RF output signals, and the input interface circuitry comprises an electrical contact positioned so as to contact a desired one of the plurality of output terminals to obtain the RF output signal corresponding thereto for use as the RF treatment signal.

14. An electrosurgical instrument according to claim 4, wherein the connector on the electrode assembly includes a plurality of contacts, and the one or more pins are jumper pins adapted to form an electrical short between a selected combination of the plurality of contacts.

15. An electrosurgical instrument according to claim 4, wherein the one or more pins cause the operation of a relay which selects a particular secondary winding or combination of secondary windings.
